(19) United States
(12) Patent Application Publication
(10) Pub. No.: US 2023/0191321 A1
(43) Pub. Date: Jun. 22, 2023

(54) AN AIR PURIFIER COMPRISING A CARBON DIOXIDE ADSORPTION UNIT

(71) Applicant: ARCELIK ANONIM SIRKETI, Istanbul (TR)

(72) Inventors: Pinar YAVUZ KAYA, Istanbul (TR); Süleyman Alper YESILÇUBUK, Istanbul (TR); Koray ERDOGAN, Istanbul (TR); Ünsal KAYA, Istanbul (TR); Sahin SIVASLIOGLU, Istanbul (TR)

(73) Assignee: ARCELIK ANONIM SIRKETI, Istanbul (TR)

(21) Appl. No.: 17/926,122

(22) PCT Filed: Jul. 8, 2021
(86) PCT No.: PCT/TR2021/050697

§ 371 Date: Nov. 17, 2022

(30) Foreign Application Priority Data
Sep. 4, 2020 (TR) 2020/14018

(51) Int. Cl. B01D 53/62; B01D 53/04; B01D 53/78

(52) U.S. Cl. CPC B01D 53/62; B01D 53/0446; B01D 53/78; B01D 2257/504; B01D 2259/4508

(57) ABSTRACT

The present invention relates to an air purifier (1) comprising a body (2) having an inlet opening (I) through which the air in the environment is sucked and an outlet opening (O) through which the cleaned air is released, and a CO₂ adsorption unit (3) which is provided on the body (2), which chemically adsorbs the carbon dioxide in the air taken into the body (2) by being supplied with air together with a basic solution and which has an inlet tube (5).

The present invention relates to an air purifier which is used in indoor environments, such as rooms, and which purifies the air.

Today, the need for ventilation and the need for fresh air become more important in environments such as homes and offices especially in high-rise buildings. Plants can be a temporary solution for daytime but when the sunlight disappears, plants also start to produce carbon dioxide like human beings and the air quality of the environment decreases. Especially in high-rise buildings, it is not always possible to continuously keep the windows open and ventilate the environment. The continuously rising carbon dioxide amount in the air adversely affects human health. The amount of oxygen in a closed environment directly affects the metabolic rate of people and the lack thereof creates weakness and fatigue in humans. Ensuring that the amount of oxygen in the closed environment does not fall below a certain level while reducing the amount of carbon dioxide generated in the environment makes the human metabolism much more vivid and vigorous.

Today, devices are known, which comprises various filters and which purifies the indoor air. However, said retain only the bacteria and dust in the air and does not adsorb the carbon dioxide in the air or purify the air.

In the state of the art Chinese Patent Document No. CN108355462, a cylindrical receptacle and a system are disclosed, used for increasing carbon dioxide adsorption in liquid-gas processes. Said system is not related to air purifiers.

The aim of the present invention is the realization of an air purifier which increases the amount of carbon dioxide adsorbed in an indoor environment (room, building, etc.) and which operates at maximum efficiency.

The air purifier of the present invention comprises an air inlet opening and a clean air outlet opening. The air purifier further comprises a base supply unit and an acid supply unit. In the air purifier, the air taken through the air inlet opening by means of a fan is delivered to the CO₂ adsorption unit together with the basic solution by means of a pump. In the CO₂ adsorption unit, the basic unit mixes with the air so as to generate bubbles, and chemically traps the carbon dioxide in the air, thus creating a carbonate compound. The bubbles formed by pumping the air into the basic liquid solution are in contact with the chemical liquid (basic solution) until reaching the liquid surface, and the reaction takes place on the bubble surface during this time. As the reaction time prolongs, the basic solution adsorbs more carbon dioxide, and converts the same to carbonate compound. Thus, this situation increases the rate of removal of carbon dioxide in the air.

The fact that said air bubbles have a higher surface area without combining with each other also increases the reaction efficiency between the air and the basic solution. Therefore, for a carbon dioxide adsorption process at maximum efficiency, it is required that the basic solution and air contact each other for as long as possible, and the bubbles formed by this contact are small and do not mix with each other.

In the CO₂ adsorption unit in the air purifier, the clean air of which the carbon dioxide is adsorbed is delivered into the indoor environment (room, etc.) through an air outlet tube, and the solutions resulting from the reaction are combined with the acid solution in another compartment by means of a base outlet tube so as to be discharged from the CO₂ adsorption unit to be neutralized. The chemicals which are neutralized by being processed with acid solution are separated into acid and base by means of an electrodialysis unit and fed into the system from the beginning. The carbon dioxide gas released as a result of the reactions is also released to the outside of the room and building through an outlet line.

The basic solution used in the air purifier of the present invention is preferably chosen from sodium hydroxide or potassium hydroxide.

In an embodiment of the present invention, the CO₂ adsorption unit is in the form of a receptacle having a lower plate covering the cylindrical body from one end and an upper plate covering the same from another end.

In the air purifier of the present invention, at least one inlet tube and a basic solution tube are provided on the lower plate. The air is pumped together with the basic solution into the CO₂ adsorption unit through the inlet tube by means of the pump.

In an embodiment of the present invention, the inlet tube and the basic solution tube are the same tube.

The air purifier of the present invention comprises the CO₂ adsorption unit having a helix member. Thus, the air-base mixture received from the inlet tube moves through the helix member so as to increase the reaction time thereof, increasing the carbon dioxide adsorption capacity.

In an embodiment of the present invention, the air purifier comprises a plurality of inlet tubes provided on the lower plate and a wall disposed between the tubes and a helix member corresponding to each inlet tube. Thus, the bubbles formed in the air-base solution pumped into the CO₂ adsorption unit cause a more efficient reaction at smaller sizes without combining with each other while the air-basic solution mixture moves for a longer time in the CO₂ adsorption unit through the helix member. In this embodiment, for example, three inlet tubes are provided on the lower plate, and the air-base mixture taken by each tube is delivered to three different helix members through separate paths by means of three walls.

In an embodiment of the present invention, the air purifier comprises the inlet tubes placed at equal intervals on the lower plate, and helix members in the same number as the number of the inlet tubes. For example, when six inlet tubes are provided on the lower plate at 60-degree intervals, the air-basic solution mixture is delivered to six different helix members from the inlet tubes. A wall is disposed between each inlet tubes.

In an embodiment of the present invention, in case there are a plurality of helix members, the lengths of the helix members can be equal to or different from each other. Thus, the reaction times of the air-basic solution mixture on each different helix member can be changed.

In the air purifier of the present invention, the upper plate of the CO₂ adsorption unit comprises an air outlet tube. The clean air of which the carbon dioxide is adsorbed is released from the air purifier by means of said tube. The clean air resulting from the reaction rises and exits through the air outlet tube on the upper plate.

In the air purifier of the present invention, the lower plate of the CO₂ adsorption unit comprises a basic solution outlet tube. The solutions resulting from the reaction move downwards through said tube due to the weight of the liquids to be transferred to be receptacle for being processed with the acid solution.

In an embodiment of the present invention, a channel provided at the center of the helix members in the CO₂ adsorption unit joins the basic solution outlet tube and the air outlet tube. By means of said channel, the liquids resulting from the reaction are discharged from the CO₂ adsorption unit with a regular flow.

In an embodiment of the present invention, the helix member is manufactured from polymeric material. Said polymeric material is preferably polypropylene. Thus, the basic solution is prevented from damaging (abrading, destroying, etc.) CO₂ adsorption unit.

By means of the present invention, an air purifier is realized, which adsorbs maximum amount of carbon dioxide in the air so as to purify the air.

The model embodiments that relate to the air purifier realized in order to attain the aim of the present invention are illustrated in the attached figures, where:

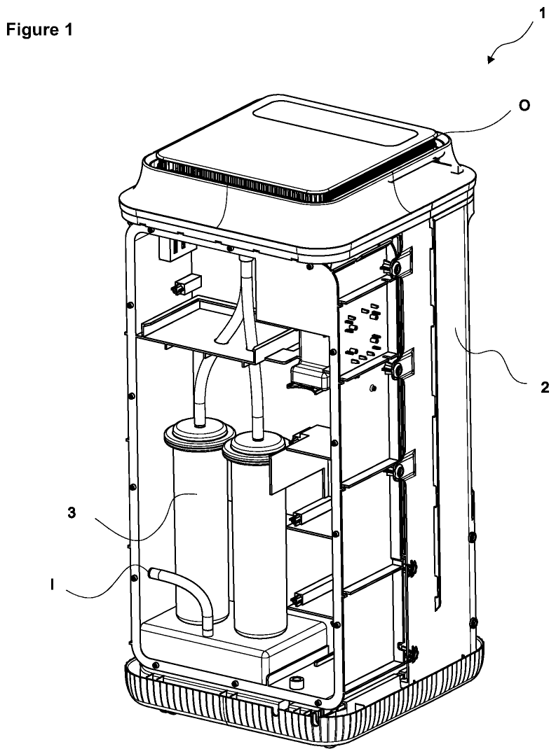

FIG. 1—is the perspective view of the air purifier in an embodiment of the present invention.

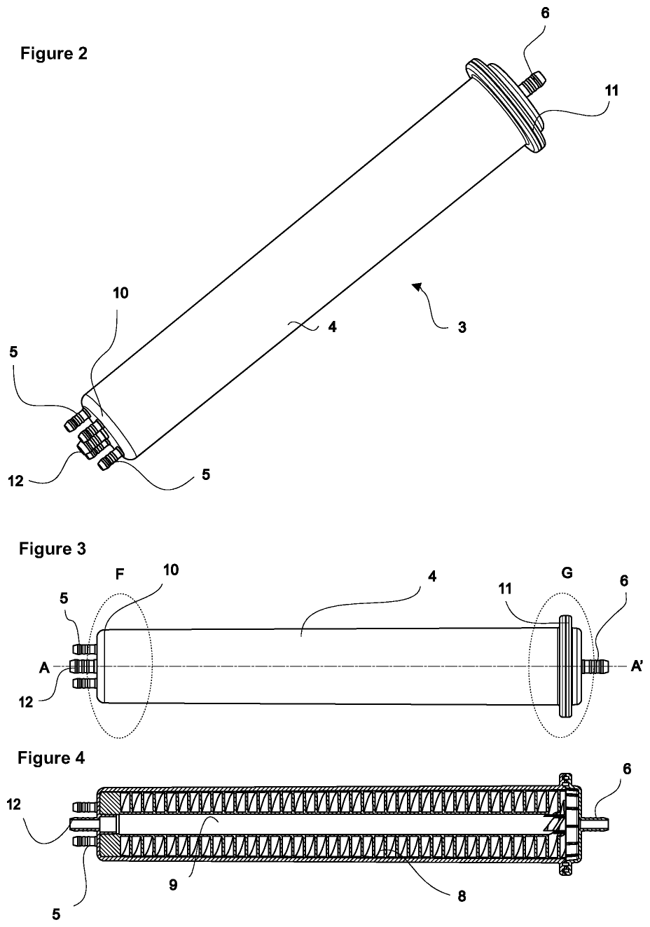

FIG. 2—is the perspective view of the CO₂ adsorption unit in the air purifier of the present invention.

FIG. 3—is the horizontal view of the CO₂ adsorption unit in the air purifier of the present invention.

FIG. 4—is the view of the cross-section A-A in FIG. 3.

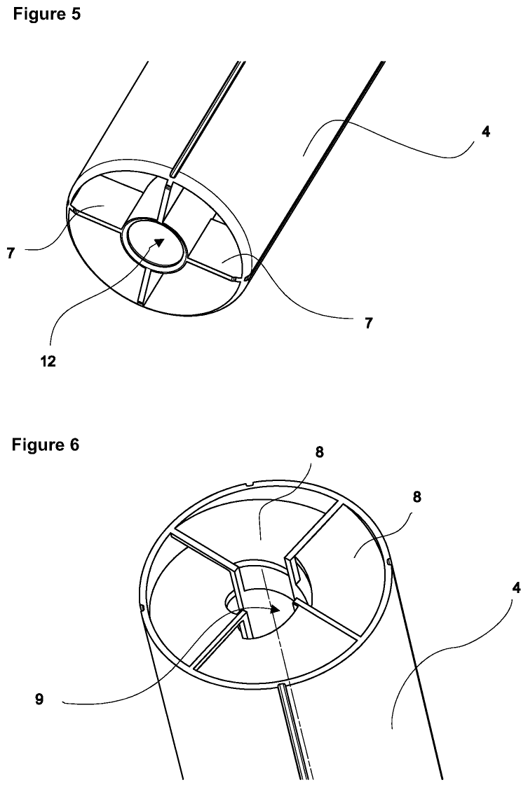

FIG. 5—is the partial view of the inner side of the F region of the CO₂ adsorption unit in FIG. 3.

FIG. 6—is the partial view of the inner side of the G region of the CO₂ adsorption unit in FIG. 3.

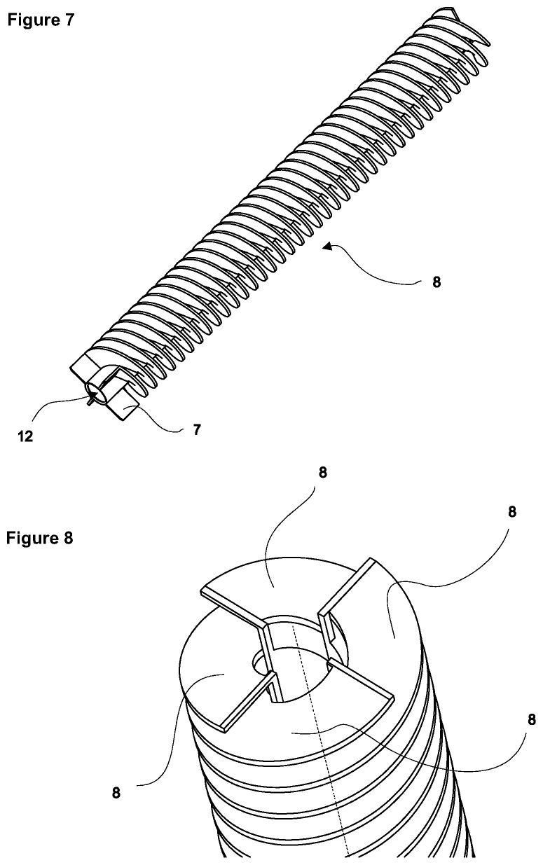

FIG. 7—is the perspective view of the helix member comprised by the CO₂ adsorption unit in the air purifier in embodiment of the present invention.

FIG. 8—is the view of the plurality of helix members in the air purifier in an embodiment of the present invention.

The elements illustrated in the figures are numbered as follows:
1. Air purifier
2. Body
3. CO₂ adsorption unit
4. Cylindrical body
5. Inlet tube
6. Air outlet tube
7. Wall
8. Helix member
9. Channel
10. Lower plate
11. Top plate
12. Basic solution outlet tube
I: Inlet opening
O: Clean air outlet opening

The air purifier (1) comprises a body (2) having an inlet opening (I) through which the air in the environment is sucked and an outlet opening (O) through which the cleaned air is released, and a CO₂ adsorption unit (3) which is provided on the body (2), which chemically adsorbs the carbon dioxide in the air taken into the body (2) by being supplied with air together with a basic solution and which has an inlet tube (5).

The air purifier (1) of the present invention comprises the CO₂ adsorption unit (3) having at least one helix member (8) forming a passageway in the CO₂ adsorption unit (3) for the basic solution-air mixture moving through the inlet tube (5).

In the air purifier (1) of the present invention, the air in the environment is taken into the body (2) by means of an inlet opening (I). The air taken into the body (2) is delivered to the CO₂ adsorption unit (3) together with a basic solution by means of a pump. The CO₂ adsorption unit (3) is a section where the air and basic solution mixture reacts, and as a result of said reaction, the carbon dioxide gas in the air in the environment is adsorbed by the base and converted into carbonate. By means of the helix member (8) of the CO₂ adsorption unit, the basic solution-air mixture contact time (reaction time) prolongs, and thus the basic solution is enabled to adsorb a higher amount of carbon dioxide.

In an embodiment of the present invention, the air purifier (1) comprises the CO₂ adsorption unit (3) having at least two inlet tubes (5), a wall (7) which is placed between the inlet tubes (5) and which separates the air-basic solution flow taken from each inlet tube (5), and at least two helix members (8) through which the air-basic solution mixture taken from each inlet tube (5) can flow without mixing with each other. Thus, the bubbles in the air-basic solution do not join with each other such that the risk of the formation of a larger bubble and decreasing the efficiency of the reaction is prevented. Moreover, by means of different inlet tubes (5), air-basic solution mixtures at different or same flow rates can be delivered to different helix members (8) through separate passageways.

In an embodiment of the present invention, the air purifier (1) comprises the CO₂ adsorption unit (3) having a cylindrical body (4) and a helix member (8) which is placed into the cylindrical body so as to contact the wall of the cylindrical body (4). In this embodiment, the helix member (8) contacts the cylindrical body (4) as a single piece or by snap-fitting, and since there is no gap therebetween, the air-basic solution mixture moves through the helix member (8) in a smooth flow.

In an embodiment of the present invention, the air purifier (1) comprises the CO₂ adsorption unit (3) having a lower plate (10) which covers the cylindrical body (4) from one end, inlet tubes (5) which are disposed onto the lower plate (10) at equal degrees, and a helix member (8) provided in a number equal to the number of inlet tubes (5). Thus, the air-basic solution mixture is delivered to the CO₂ adsorption unit (3) in a smooth flow from each inlet tube (5), ensuring a more efficient reaction.

In an embodiment of the present invention, the air purifier (1) comprises the CO₂ adsorption unit (3) having a helix member (8) which has a length almost equal to the length of the cylindrical body (4). Thus, the path the air-basic solution mixture follows in the helix member (8) starting from the inlet tube (5) is extended, increasing the efficiency of the reaction.

In an embodiment of the present invention, the air purifier (1) comprises the CO₂ adsorption unit (3) having an upper plate (11) which covers the cylindrical body (4) from another end, and at least one air outlet tube (6) which is disposed on the upper plate (11). Thus, the clean air of which the carbon dioxide is adsorbed rises and flows towards the clean air outlet opening (O) on the body (2) through the air outlet tube (6) without requiring any extra guidance (pump, etc.).

In an embodiment of the present invention, the air purifier (1) comprises a basic solution outlet tube (12) which is provided on the lower plate (10) and a channel (9) which connects the basic solution outlet tube (12) and the air outlet tube (6) and which is disposed at the center of the helix member (8). Thus, the solutions remaining and forming as a result of the air-basic solution reaction are quickly transferred from the CO₂ adsorption unit (3), without entering the helix member (8) again, to another receptacle to be processed with acid to be neutralized.

In an embodiment of the present invention, the air purifier (1) comprises the CO2 adsorption unit (3) having a helix member (8) which is manufactured from a polymeric material, preferably from polypropylene. Thus, the basic solution is prevented from damaging the CO₂ adsorption unit (3).

By means of the present invention, an air purifier (1) is realized, wherein the reaction efficiency and time are increased and a higher amount of carbon dioxide in the air is adsorbed.

1. An air purifier comprising a body having an inlet opening through which the air in the environment is sucked and an outlet opening through which the cleaned air is released, and a CO₂ adsorption unit which is provided on the body, which chemically adsorbs the carbon dioxide in the air taken into the body by being supplied with air together with a basic solution and which has an inlet tube, characterized by the CO₂ adsorption unit having at least one helix member forming a passageway in the CO₂ adsorption unit for the basic solution-air mixture moving through the inlet tube.

2. An air purifier as in claim 1, characterized by the CO₂ adsorption unit having at least two inlet tubes, a wall which is placed between the inlet tubes and which separates the air-basic solution flow taken from each inlet tube, and at least two helix members through which the air-basic solution mixture taken from each inlet tube can flow without mixing with each other.

3. An air purifier as in claim 1, characterized by the CO₂ adsorption unit having a cylindrical body and a helix member which is placed into the cylindrical body so as to contact the wall of the cylindrical body.

4. An air purifier as in claim 3, characterized by the CO₂ adsorption unit having a lower plate which covers the cylindrical body from one end, inlet tubes which are disposed onto the lower plate at equal degrees, and a helix member provided in a number equal to the number of inlet tubes.

5. An air purifier as in claim 3, characterized by the CO₂ adsorption unit having a helix member which has a length almost equal to the length of the cylindrical body.

6. An air purifier as in claim 3, characterized by the CO₂ adsorption unit having an upper plate which covers the cylindrical body from another end, and at least one air outlet tube which is disposed on the upper plate.

7. An air purifier as in claim 4, characterized by a basic solution outlet tube which is provided on the lower plate and a channel which connects the basic solution outlet tube and the air outlet tube and which is disposed at the center of the helix member.

8. An air purifier as in claim 1, characterized by the CO₂ adsorption unit having a helix member which is manufactured from a polymeric material, preferably from polypropylene.

9. An air purifier as in claim 4, characterized by the CO₂ adsorption unit having an upper plate which covers the cylindrical body from another end, and at least one air outlet tube which is disposed on the upper plate.

10. An air purifier as in claim 5, characterized by the CO₂ adsorption unit having an upper plate which covers the cylindrical body from another end, and at least one air outlet tube which is disposed on the upper plate.

11. An air purifier as in claim 5, characterized by a basic solution outlet tube which is provided on the lower plate and a channel which connects the basic solution outlet tube and the air outlet tube and which is disposed at the center of the helix member.

12. An air purifier as in claim 6, characterized by a basic solution outlet tube which is provided on the lower plate and a channel which connects the basic solution outlet tube and the air outlet tube and which is disposed at the center of the helix member.

13. An air purifier as in claim 2, characterized by the CO₂ adsorption unit having a helix member which is manufactured from a polymeric material, preferably from polypropylene.

14. An air purifier as in claim 3, characterized by the CO₂ adsorption unit having a helix member which is manufactured from a polymeric material, preferably from polypropylene.

15. An air purifier as in claim 4, characterized by the CO₂ adsorption unit having a helix member which is manufactured from a polymeric material, preferably from polypropylene.

16. An air purifier as in claim 5, characterized by the CO₂ adsorption unit having a helix member which is manufactured from a polymeric material, preferably from polypropylene.

17. An air purifier as in claim 6, characterized by the CO₂ adsorption unit having a helix member which is manufactured from a polymeric material, preferably from polypropylene.

18. An air purifier as in claim 7, characterized by the CO₂ adsorption unit having a helix member which is manufactured from a polymeric material, preferably from polypropylene.
